SEQUENCE LISTING (2 sequences)

SEQ ID NO: 1 - DNA, 19 bp, Artificial Sequence
  forward primer
gagtttgatcctggctcag

SEQ ID NO: 2 - DNA, 16 bp, Artificial Sequence
  reverse primer
taccttgttacgactt

(19) United States
(12) Patent Application Publication
(10) Pub. No.: US 2006/0211105 A1
(43) Pub. Date: Sep. 21, 2006

(54) Rhodococcus erythropolis LG12 having acrylic acid degrading activity and method for removing acrylic acid using the same

(76) Inventors: Sanghyun Lee, Daejeon (KR); Junhyeong Cho, Daejeon (KR); Ohjin Park, Daejeon (KR); Joowon Rhee, Daejeon (KR); Si Jae Park, Daejeon (KR)

Correspondence Address: MCKENNA LONG & ALDRIDGE LLP, 1900 K STREET, NW, WASHINGTON, DC, 20006, US

(21) Appl. No.: 11/224,314

(22) Filed: Sep. 13, 2005

(30) Foreign Application Priority Data
Sep. 15, 2004 (KR) 10-2004-0073916

(51) Int. Cl. C12N 1/20; B09C 1/10

(52) U.S. Cl. 435/262.5; 435/252.1

(57) ABSTRACT

The present invention relates to a novel strain, Rhodococcus erythropolis LG12, having acrylic acid degrading activity and resistance to acrylic acid, and a method for removing acrylic acid from acrylic acid-containing contaminants using the same. The strain according to the invention has not only acrylic acid degrading activity but also resistance to a high concentration of acrylic acid thus will be useful to remove acrylic acid from acrylic acid-containing contaminants.

### CROSS-REFERENCE TO RELATED APPLICATION

This application claims the benefit of filling date of Korean Patent Application No. 10-2004-0073916 filed on Sep. 15, 2004, in the Korean Intellectual Property Office, the disclosure of which is incorporated herein in its entirety by reference.

### BACKGROUND OF THE INVENTION

1. Field of the Invention

The present invention relates to a novel strain, Rhodococcus erythropolis LG12, having acrylic acid degrading activity and resistance to acrylic acid, and a method for removing acrylic acid from acrylic acid-containing contaminants using the same.

2. Background of the Related Art

As known in the prior art, microorganisms degrading acrylic acid include bacteria such as Pseudomonas sp. and molds such as Geotrichum sp., Trichoderma sp., Candida rugosa and Byssochlamys sp. It is known that bacteria grow under relatively simple conditions and it is easy to culture bacteria, but cannot grow at high acrylic acid concentration, for example, a concentration of more than 10 g/L (1 wt %). For example, Pseudomonas sp. shows suppressed growth at an acrylic acid concentration of more than 1.5 g/L (Shanker, R., Arch. Microbiol., 154:192, 1990; Bringmann & Kuhn, Water Research, 14:231, 1980).

Molds are known to grow at relatively high acrylic acid concentrations. For example, Geotrichum sp. and Trichoderma sp. are known to degrade 1 wt % of acrylic acid within 10 days (Heena Dave, Biotechnology letters, 18:963, 1996). Also, it is known that Candida rugosa degrades 2 wt % of acrylic acid within 4 days (Hasegawa, J., J Ferment. Technol., 60:591, 1992), and Byssochlamys sp. degrades 7 wt % of acrylic acid within 14 days (Kazuhiro Takamizawa, Appl. Microbiol. Biotech., 40:196, 1993). The degradation of acrylic acid by these molds has shortcomings in that it requires a relatively long degradation time, complex conditions for strain growth, and a long culture time. Another shortcoming is that it is not easy to prepare recombinant strains of molds because transformation methods for molds are not established.

Meanwhile, Rhodococcus sp. is gram-positive bacteria and belongs to nocardioform Actinomycetes. In the prior art, Rhodococcus sp. was used to convert a nitrile compound, such as acrylonitrile, to its acid, such as acrylic acid. For example, U.S. Pat. No. 5,135,858 discloses a process of using Rhodococcus rhodochrous J-1 to convert a nitrile compound, such as acrylonitrile, to its acid, such as acrylic acid. However, acrylic acid is accumulated in medium and not degraded by Rhodococcus rhodochrous J-1. Accordingly, Rhodococcus sp. microorganisms degrading acrylic acid have not been known.

As a result, there still remains a demand for microorganisms which require simple conditions for growth as in the case of bacteria, rapidly grow, have resistance to acrylic acid and can rapidly degrade acrylic acid. Accordingly, the present inventors have made an effort to find a strain capable of degrading acrylic acid using acrylic acid as a carbon source, and as a result, have discovered a strain belonging to Rhodococcus erythropolis, which can grow even at a high concentration of acrylic acid and rapidly degrade acrylic acid, thereby completing the present invention.

### SUMMARY OF THE INVENTION

Therefore, it is an object of the present invention to provide a strain belonging to Rhodococcus erythropolis, which has acrylic acid degrading activity and resistance to a high concentration of acrylic acid.

Another object of the present invention is to provide a method for removing acrylic acid from acrylic acid-containing contaminants using said strain.

To achieve the above objects, in one aspect, the present invention provides an acrylic acid-resistant Rhodococcus erythropolis strain having acrylic acid degrading activity.

In another aspect, the present invention provides a method for removing acrylic acid from an acrylic acid-containing contaminant, the method comprising the steps of: mixing the acrylic acid-containing contaminant with the Rhodococcus erythropolis strain or its culture broth; and culturing the mixture.

The above and other features and embodiments of the present invention will be more fully apparent from the following detailed description and appended claims.

### BRIEF DESCRIPTION OF THE DRAWINGS

FIG. 1 shows a scanning electron microscope photograph of the 12 HPL strain isolated according to the present invention.

FIG. 2 shows a phylogenetic tree obtained by the search for homology with the 1,454 bp nucleotide sequence of 16S rRNA of the 12HPL strain isolated according to the present invention.

FIG. 3 shows the acrylic acid degrading activity of the inventive Rhodococcus erythropolis LG12 strain in a medium containing each of 1 wt %, 2 wt % and 4 wt % of acrylic acid.

FIG. 4 shows measurement results for the remaining amount of each of acrylic acid, crotonic acid, metacrylic acid and 2-chloroacrylic acid after adding each of the acids to a culture medium of the inventive Rhodococcus erythropolis LG 12 strain.

### DETAILED DESCRIPTION OF THE PREFERRED EMBODIMENT

In one aspect, the present invention relates to an acrylic acid-resistant Rhodococcus erythropolis strain having acrylic acid degrading activity. In the present invention, the strain has deposition number of KCTC 18102P.

The inventive microorganism, which is a strain belonging to Rhodococcus erythropolis has resistance to acrylic acid and has activity to degrade acrylic acid. The inventive strain has remarkably high acrylic acid degrading activity, compared to microorganisms belonging to other species, for example, Escherichia sp., Pseudomonas sp., Bacillus sp., and Candida sp. Also, the inventive strain has resistance to acrylic acid and can grow even at a high concentration of acrylic acid. The inventive strain can grow even at an acrylic acid concentration of less than 5% by weight, and preferably 1˜4% by weight.

As used herein, the term “acrylic acid degrading activity” means that the strain can assimilate or dissimilate acrylic acid using acrylic acid as a carbon source in a medium containing acrylic acid so as to reduce the concentration of acrylic acid in the medium.

In another aspect, the present invention relates to a method for removing acrylic acid from an acrylic acid-containing contaminant, the method comprising the steps of: mixing the acrylic acid-containing contaminant with the Rhodococcus erythropolis strain or its culture broth; and culturing the mixture.

In the inventive method, the culture conditions of the strain may be the same as known in the art except that acrylic acid is contained in the medium. As the acrylic acid-containing contaminant, any contaminant may be used if it contains acrylic acid. Examples of the acrylic acid-containing contaminant include acrylic acid itself, and a solution, sewage, wastewater, and the like, containing acrylic acid.

Hereinafter, the present invention will be described in more detail by examples. It is to be understood, however, that these examples are for illustrative purpose only and are not intended to limit the scope of the present invention.

### EXAMPLE 1

### Isolation of Microorganism Using Acrylic Acid as Carbon Source from the Soil

Soil around the highway in Jeongeup-shi, Jeollabuk-do, Korea, was collected and dried at room temperature for 24 hours. 1.0 g of the dried soil sample was added into a 50 ml baffle flask containing 20 ml of a liquid medium (see Tables 1 and 2; also the same in the following procedure) containing 5 ml of acrylic acid (2 mM) and cultured in a shaking incubator at 200 rpm and 30° C. for 3 days. Then, 1 ml of the culture broth was added to 20 ml of a fresh liquid medium and cultured in the same conditions as above. This procedure was repeated three times, and then, the culture was spread on an LB plate medium and incubated at 30° C. After completion of the culture, a novel strain using acrylic acid as a carbon source was isolated from the colony of the LB plate medium and named “12HPL”.

TABLE 1
Composition of liquid medium containing acrylic acid

| Components | Content (per L) | |
|---|---|---|
| K₂HPO₄ | 7 | g |
| KH₂PO₄ | 3 | g |
| NH₄Cl | 1 | g |
| NaCl | 0.5 | g |
| MgSO4 solution (200 g/L) | 1 | ml |
| Vitamin solution | 10 | ml |
| Metal solution (0.1 g/L) | 10 | ml |

TABLE 2
Composition of trace metal solution and vitamin solution

| | Components | Content (per L) | |
|---|---|---|---|
| Trace metal | Na₂B₄O₇.10H₂O | 100 | mg |
| solution | CoCl₂.6H₂O | 20 | mg |
| | CuSO₄.6H₂O | 10 | mg |
| | NiCl.H₂O | 10 | mg |
| | Na₂MoO₄.2H₂O | 10 | mg |
| | CaCl₂.2H₂O | 10 | mg |
| | MnSO₄.5H₂O | 100 | mg |
| | FeSO₄.7H₂O | 200 | mg |
| Vitamin solution | thiamine.HCl | 4 | mg |
| | riboflavin | 2 | mg |
| | D-pantothenic acid | 4 | mg |
| | pyridoxin.HCl | 4 | mg |
| | p-amonibenzoic acid | 4 | mg |
| | nicotinic acid | 4 | mg |
| | inocitol | 20 | mg |
| | biotin (0.02% solution) | 100 | μl |

When acrylic acid was added to the liquid medium, the medium was neutralized with calcium hydroxide (Ca(OH)₂) and filtered through a 0.22 μm filtration membrane before introduction.

### EXAMPLE 2

### Identification of Isolated 12HPL Strain

In this Example, the 12HPL strain isolated in Example 1 was identified by electron microscopic and molecular biological methods.

(1) Morphological Characteristics by Electron Microscope

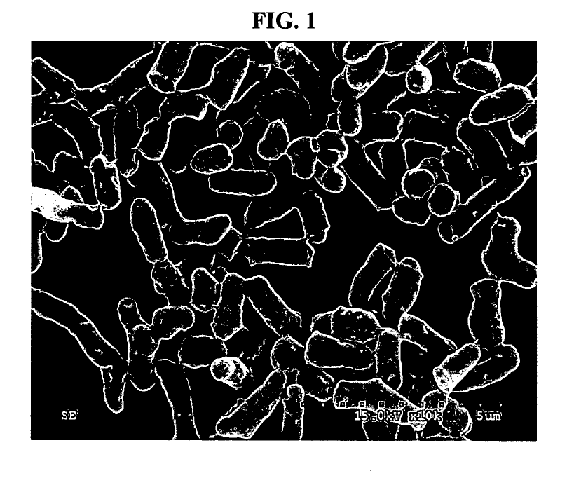

The 12HPL strain was first spread on an LB plate medium and incubated at 30° C. for 3 days. Then, the culture was observed for morphological characteristics with a scanning electron microscope (FIG. 1). FIG. 1 shows a scanning electron microscope photograph of the 12HPL strain. As shown in FIG. 1, the 12HPL strain had a long cylindrical shape which is a characteristic observed commonly in Rhodococcus sp. known in the prior art.

(2) Classification by Homology Comparison With 16S rRNA Nucleotide Sequence

First, a chromosome was isolated from the 12HPL strain using Wizard Genomic DNA Purification Kit (Promega Co., Catalogue No: A1120). Then, 16S rRNA was amplified by PCR using the isolated chromosome as a template with primers HK 12 (SEQ ID NO: 1) and HK13 (SEQ ID NO: 2) (Qiong Cheng, J Bacteriol., 182:4744, 2000). The PCR reaction was performed in the following conditions: addition of the HK12 primer, initial denaturation at 95° C. for 5 min, 10 cycles of each consisting of denaturation at 95° C. for 1 min, annealing at 50° C. for 1 min and polymerization at 72° C. for 1 min and 30 sec, and then, addition of the HK13 primer, followed by 30 cycles of each conducted in the same conditions as described above. The PCR reaction mixture was added with 200 μM dNTP, 1.5 mM MgCl₂, 10 μl 10× buffer, 50 ng template DNA, 5 units of Taq polymerase, 0.1 unit of pfu polymerase, 20 pmole of each of the primers, and water to a final volume of 100 μl.

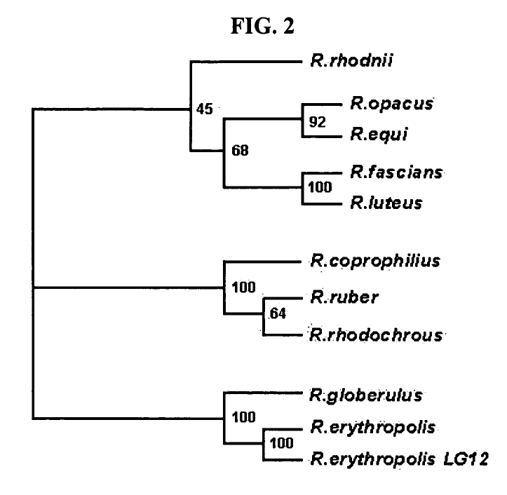

The obtained 16S rRNA PCR product was inserted into a pGEM T-easy vector (Promega Co.), and then, the 1,454 bp nucleotide sequence of 16S rDNA from the recombinant pGEM T-easy vector was determined. Next, the phylogenetic tree of the 1,454 bp nucleotide sequence was analyzed using Clustal X™ program (FIG. 2). FIG. 2 shows a phylogenetic tree obtained by the homology search between the 12HPL strain in the present invention and the 1,454 bp nucleotide sequence of 16S rRNA. As shown in FIG. 2, the 12HPL strain isolated in the present invention was determined to have a homology of more than 99% with the Rhodococcus erythropolis strain.

The results of the electron microscopic analysis of morphological characteristics and the phylogenetic classification by the search of homology with 16S rRNA suggest that the inventive 12HPL strain is a novel strain which belongs to Rhodococcus erythropolis, assimilates acrylic acid and has resistance to acrylic acid. As a result, the inventive 12HPL strain was named “Rhodococcus erythropolis LG12” and deposited under accession number KCTC 18102P on May 19, 2004 in the Korean Collection for Type Cultures (KCTC), an International Depositary Authority.

### EXAMPLE 3

### Measurement of Acrylic Acid Degrading Activity of Rhodococcus Erythropolis LG12

In this Example, the acrylic acid degrading activity of Rhodococcus erythropolis LG12 was analyzed, comparing with that of other various microorganisms.

The colony of each microorganism cultured on an LB plate medium was inoculated and cultured in 3 ml of a YEPD liquid medium (10 g/L yeast extract, 20 g/L bactopeptone, 20 g/L glucose). 0.3 ml of the obtained culture was added into a 15 ml disposable tube (Falcon Co.) containing 3 ml of a YEPD liquid medium and cultured in a shaking incubator at 200 rpm and 30° C. for 2 days. Then, acrylic acid was added to the medium to a final concentration of 1%, and after 1 day, the concentration of remaining acrylic acid was analyzed by high-performance liquid chromatography (HPLC) (Waters, Inc.). A mobile phase used in the liquid chromatography was a 7:3 mixture of water and acetonitrile, the flow rate of solvent was 1 ml/min, a Capcel PAK C18 column was used, and detection was carried out at 210 nm. Analysis results for the acrylic acid degrading activity of the microorganisms are shown in Table 3 below.

In Table 3, the acrylic acid degrading activities of various microbial strains are shown relative to the activity of the inventive Rhodococcus erythropolis LG12 strain taken as 100%. As shown in Table 3, the Rhodococcus erythropolis LG12 strain has remarkably high acrylic acid degrading activity, compared to not only microorganisms belonging to other genera, such as Pseudomonas, Candida, Escherichia, and Bacillus, but also other species of the genus Rhodococcus.

TABLE 3
Acrylic acid degrading activities of Rhodococcus erythropolis
LG12 and other various strains

| Strains | Relative activity (%) |
|---|---|
| Rhodococcus rhodochrous | 58.6 |
| Rhodococcus globerulus | 3.4 |
| Rhodococcus zopfi | 1.4 |
| Rhodococcus equi | 10.3 |
| Rhodococcus rhodnii | 2.1 |
| Rhodococcus rubber | 4.8 |
| Rhodococcus erthropolis LG12 | 100 |
| Pseudomonas pavonacea | 6.9 |
| Pseudomonas cepacia | 2.1 |
| Pseudomonas aeruginosa | 1.4 |
| Candida rugosa | 69 |
| Escherichia coli | 0.7 |
| Bacillus cereus | 0.7 |

### EXAMPLE 4

### Measurement of Acrylic Acid Degrading Activity of Rhodococcus Eruthropolis LG12

In this Example, the acrylic acid degrading activity of the Rhodococcus erythropolis LG12 strain was measured in various conditions.

First, the inventive Rhodococcus erythropolis LG12 strain was allowed to grow on a YEPD solid medium to obtain a single colony. The colony was added into a 15 ml culture tube (Falcon Co.) containing 3 ml of a YEPD liquid medium (10 g/L yeast extract, 20 g/L bactopeptone, 20 g/L glucose) and cultured in a shaking incubator (Jeio Tech Co., Ltd.) at 200 rpm and 30° C. for 1-2 days. When the OD₆₀₀ of the medium reached 30, acrylic acid was added to the medium to final concentrations of 1 wt %, 2 wt % and 4 wt %, and the culture was continued while the remaining amount of acrylic acid was analyzed at each reaction time.

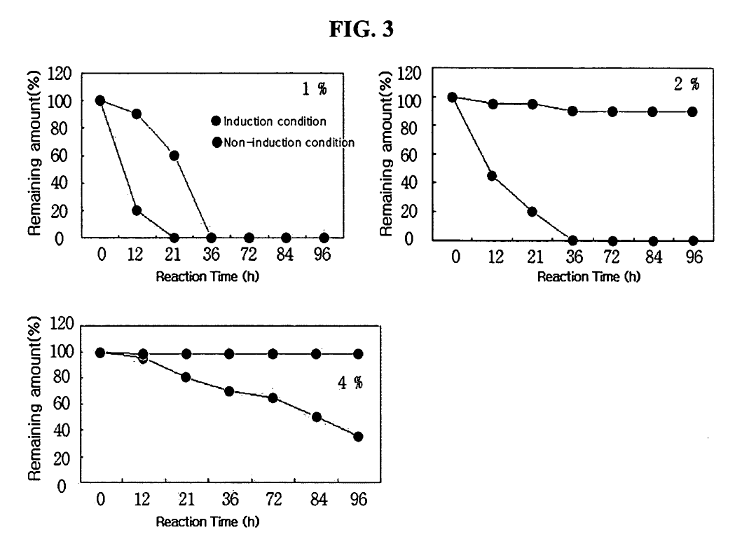

Also, in order to examine the induction effect of acrylic acid on the acrylic acid degrading activity of the strain, when the growth of the strain during culture in a YEPD liquid medium reached an OD₆₀₀ of 15, acrylic acid was added to the culture medium to a final concentration of 0.1 wt %. Then, the culture was continued until the growth of the strain reached an OD₆₀₀ of 30. In the culture procedure, other conditions were the same as described above. The analysis of acrylic acid was carried out using HPLC in the same manner as in Example 3 (FIG. 3).

FIG. 3 shows the acrylic acid degrading activity of Rhodococcus erythropolis LG12 in a medium containing each of 1 wt %, 2 wt % and 4 wt % of acrylic acid. As shown in FIG. 3, the inventive strain showed the induction effect of increasing the degradation of acrylic acid as a result of the addition of acrylic acid during the culture of the strain, had resistance even to 4 wt % of acrylic acid and could degrade 4 wt % of acrylic acid within about 4 days.

### EXAMPLE 5

### Examination of Specificity of Rhodococcus Erythropolis LG12 to Acrylic Acid Degrading Activity

In this Example, it was examined whether the inventive Rhodococcus erythropolis LG12 strain specifically degrades acrylic acid among various compounds.

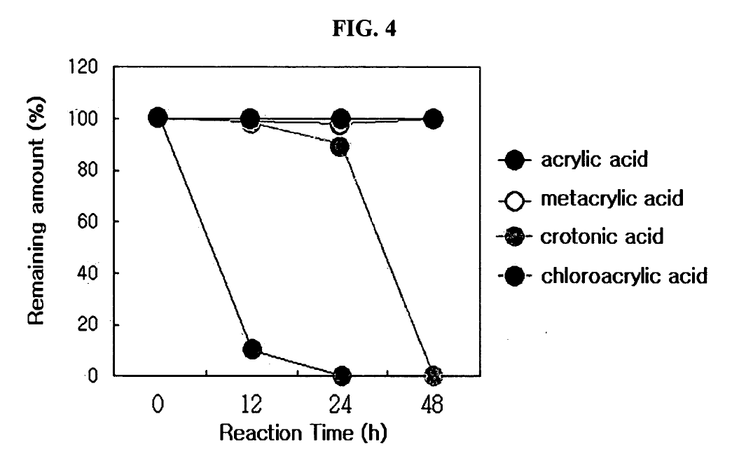

The inventive Rhodococcus erythropolis LG12 strain was allowed to grow on a YEPD solid medium to obtain a single colony. Then, the single colony was inoculated into a 15 ml culture tube (Falcon Co.) containing 3 ml of a YEPD liquid medium (10 g/L yeast extract, 20 g/L bactopeptone, and 20 g/L glucose) and cultured in a shaking incubator (Jeio Tech Co., Ltd.) at 200 rpm and 30° C. until an OD₆₀₀ of 30 was reached. Then, each of acrylic acid, crotonic acid, metacrylic acid and 2-chloroacrylic acid was added to the culture medium to a final concentration of 0.5% by weight. After the addition, the concentrations of the remaining compounds were measured at given intervals. The concentrations of the compounds were measured using HPLC in the same manner as in Example 3 (FIG. 4).

FIG. 4 shows the remaining amount of each of acrylic acid, crotonic acid, metacrylic acid and 2-chloroacrylic acid after adding each of the acids to the culture broth of the inventive Rhodococcus erythropolis LG12 strain. As could be seen in FIG. 4, the remaining amount of acrylic acid among the compounds having similar chemical structures was specifically and significantly reduced. This result indicates that the inventive Rhodococcus erythropolis LG12 strain specifically degrades acrylic acid.

As described in detail above, the Rhodococcus erythropolis LG 12 strain according to the present invention has not only acrylic acid degrading activity but also resistance to a high concentration of acrylic acid thus will be useful to effectively remove acrylic acid from acrylic acid-containing contaminants.

While the present invention has been described in detail with reference to the specific features, it will be apparent to those skilled in the art that this description is only for a preferred embodiment and does not limit the scope of the present invention. Thus, the substantial scope of the present invention will be defined by the appended claims and equivalents thereof.

1. An acrylic acid-resistant Rhodococcus erythropolis strain having acrylic acid degrading activity

2. The acrylic acid-resistant Rhodococcus erythropolis strain according to claim 1, wherein the strain has Korean Collection for Type Cultures accession number KCTC 10838BP.

3. The acrylic acid-resistant Rhodococcus erythropolis strain according to claim 1, wherein the strain can grow at an acrylic acid concentration of 1˜5 wt. %.

4. A method for removing acrylic acid from an acrylic acid-containing contaminant, the method comprising the steps of: mixing the acrylic acid-containing contaminant with the Rhodococcus erythropolis strain according to claim 1 or its culture broth; and culturing the mixture.

5. The method for removing acrylic acid from an acrylic acid-containing contaminant of claim 4, wherein the acrylic acid-containing contaminant is sewage or wastewater containing acrylic acid.

6. A method for removing acrylic acid from an acrylic acid-containing contaminant, the method comprising the steps of: mixing the acrylic acid-containing contaminant with the Rhodococcus erythropolis strain according to claim 2 or its culture broth; and culturing the mixture.

7. The method for removing acrylic acid from an acrylic acid-containing contaminant of claim 6, wherein the acrylic acid-containing contaminant is sewage or wastewater containing acrylic acid.

8. A method for removing acrylic acid from an acrylic acid-containing contaminant, the method comprising the steps of: mixing the acrylic acid-containing contaminant with the Rhodococcus erythropolis strain according to claim 3 or its culture broth; and culturing the mixture.

9. The method for removing acrylic acid from an acrylic acid-containing contaminant of claim 8, wherein the acrylic acid-containing contaminant is sewage or wastewater containing acrylic acid.
